(19) United States
(12) Patent Application Publication
(10) Pub. No.: US 2014/0075673 A1
(43) Pub. Date: Mar. 20, 2014

(54) Patient Support Device For Use In An Imaging Machine

(71) Applicants: James E. Weedling, Center Valley, PA (US); Robert E. Weedling, Delray Beach, FL (US)

(72) Inventors: James E. Weedling, Center Valley, PA (US); Robert E. Weedling, Delray Beach, FL (US)

(73) Assignee: Airpal Inc., Coopersburg, PA (US)

(21) Appl. No.: 13/620,006

(22) Filed: Sep. 14, 2012

(51) Int. Cl. A61B 6/04; A47C 27/08

(52) U.S. Cl. 5/601

(57) ABSTRACT

A patient support device for use in conjunction with a pallet of an imaging machine, including an inflatable mattress having a plurality of adjacent interconnected transverse air chambers and a mechanism for controlled venting of the air chambers, a sheet member disposed across a top of the mattress and extending laterally outward with respect to the transverse air chambers, the sheet member defining a patient support surface, and at least one longitudinal strip of friction enhancing material attached to a bottom surface of the inflatable mattress to inhibit sliding of the inflatable mattress on a support surface.

### BACKGROUND

It is often very difficult for medical personnel to move and position a patient, particularly an obese patient with limited mobility, in a medical imaging machine such as an MRI machine, a CAT-scan machine, and the like. And while patient transfer mattresses are known, for example as disclosed in U.S. Pat. No. 5,561,873, those transfer mattresses are capable of delivering a patient to a position adjacent to the imaging machine but are not configured to manipulate a patient onto a pallet, or while on the pallet, of the imaging machine itself.

Further, there is no existing device capable of gently transferring a patient from a gurney or other flat surface positioned adjacent to an imaging onto the concave bed or pallet of the imaging machine. In particular, it can be very difficult to position a large or obese patient in the desired position in the concave imaging machine pallet. Moreover, it can be extremely difficult to transfer a large or obese patient off of the pallet and back onto a gurney, since the transfer includes raising the patient from the concave pallet to a level matching that of the gurney.

### SUMMARY

An embodiment of a patient support device is disclosed for use in conjunction with a pallet of an imaging machine. The device includes an inflatable mattress or pad having a plurality of adjacent interconnected transverse air chambers and a microperforated bottom surface, a sheet member disposed across a top of the mattress and extending laterally outward with respect to the transverse air chambers, the sheet member defining a patient support surface, and at least one longitudinal strip of friction enhancing material attached to the bottom surface of the inflatable mattress to inhibit sliding of the inflatable mattress. As an alternative to microperforation, the bottom surface of the inflatable mattress may have another mechanism for controlled venting of the air chambers. For example, the bottom surface of the inflatable mattress can be partially or entirely perforated or porous. As another alternative, the bottom surface of the inflatable mattress can be nonporous and nonperforated, and a check valve or other pressure control mechanism can be used to maintain inflation of the mattress and to selectively allow air to be passively or actively removed for deflation.

When the imaging machine includes a rigid base member configured to mate with the pallet, the mattress can be used in conjunction with the rigid base member, which may have a concave surface, a substantially flat surface, or a surface having a shape other than concave or flat. To secure the patient support device to a rigid base member, the device may further include a plurality of straps each fastened to opposite edges of the sheet member and extending beneath the bottom surface of the inflatable mattress.

The sheet member may include a sleeve for positioning an accessory beneath the patient support surface. Alternatively, an accessory may be attached to the top of the patient support surface of the sheet member using fasteners. The fasteners may include, but are not limited to, snaps, buttons, hook-and-loop fasteners, and zippers. The accessory can include a therapeutic pad, a cushion, or similar to provide support for a patient. The sleeve and/or fasteners can also be configured to receive a flexible, semi-rigid board adapted to flex between a generally flat shape and a generally curved shape about a longitudinal axis of the patient support surface.

To enhance stability of the patient support device, the inflatable mattress may further include one or more longitudinal air chambers interconnected with the transverse air chambers and extending longitudinally along at least a portion of either side of the inflatable mattress. In one embodiment, a pair of longitudinal air chambers is provided, with one air chamber on one side of the transverse air chambers and the other air chamber on the other side of the transverse air chambers.

Another embodiment of a patient support device is disclosed for use in conjunction with a pallet of an imaging machine and a rigid base member configured to mate with the pallet. The device includes an inflatable mattress or pad having a plurality of adjacent interconnected transverse air chambers and a microperforated bottom surface, a sheet member disposed across a top of the mattress and extending laterally outward with respect to the transverse air chambers, the sheet member defining a patient support surface, the sheet member including a sleeve for positioning an accessory beneath the patient support surface, two longitudinal strips of friction enhancing material attached to the bottom surface of the inflatable mattress to inhibit sliding of the inflatable mattress, and a plurality of straps each fastened to opposite edges of the sheet member and extending beneath the bottom surface of the inflatable mattress for securing the patient support device to the rigid base member.

While the rigid base member is preferably concave or includes an upper surface that is at least partially concave, in some cases the rigid base member is substantially flat or has a shape other than concave or flat. In one variation, instead of a sleeve, fasteners may be provided for attaching an accessory on top of the patient support surface.

In another variation, instead of a microperforated bottom surface, the transverse air chambers may have a substantially air-tight bottom surface, and a valve or other pressure control mechanism may be provided for controlled release of air from the inflatable mattress.

In yet another variation, instead of using straps fastened to the sheet member, the device may be secured or substantially prevented from moving with respect to the rigid base member by another mechanism, such as a transverse attachment member extending beneath the base member and having a non-stick central portion bounded on either side by attachment portions configured to mate in engagement with features on the bed of the imaging device.

The accessory may include one or more therapeutic pads and/or support cushions. The sleeve and/or fasteners can also be configured to receive a flexible, semi-rigid board adapted to flex between a generally flat shape and a generally curved shape about a longitudinal axis of the patient support surface.

An embodiment of a method is disclosed for positioning a patient on a pallet of an imaging machine using a patient support device. The method includes positioning a patient support device the pallet or on a concave rigid base member configured to mate with the pallet. The device for use with this method includes an inflatable mattress having a plurality of adjacent interconnected transverse air chambers and a microperforated bottom surface; a sheet member disposed across a top of the mattress and extending laterally outward with respect to the transverse air chambers, the sheet member defining a patient support surface; and at least one longitudinal strip of friction enhancing material attached to the bottom surface of the inflatable mattress to inhibit sliding of the inflatable mattress. The device may also include a plurality of straps each fastened to opposite edges of the sheet member and extending beneath the bottom surface of the inflatable mattress for securing the patient support device to the base member of the imaging machine.

The method further includes supplying a flow pressurized air to the inflatable mattress to cause the patient support surface to be maintained in a generally flat condition; positioning a patient on top of the patient support surface while continuing to supply the flow of pressurized air at a sufficient level to maintain the patient support surface in a generally flat condition; and decreasing the flow of pressurized air while allowing air to escape from the microperforated bottom surface of the mattress such that, under the weight of the patient, the patient support surface is lowered so as to generally conform to the shape of the pallet, thereby lowering the patient into position for imaging.

The method may further include increasing the flow of pressurized air to overcome the air escaping from the microperforated bottom surface of the mattress such that the patient support surface is raised to a generally flat condition, thereby supporting the patient above the pallet.

### BRIEF DESCRIPTION OF DRAWINGS

FIG. 1 is a top perspective view showing an embodiment of a patient support device for use in an imaging machine.

FIG. 2 is a bottom view of showing an embodiment of a patient support device for use in an imaging machine without a base member installed.

FIG. 3 is an end view of the embodiment of a patient support device for use in an imaging machine of FIG. 2.

FIGS. 4 and 5 are top and side perspective views showing an embodiment of a patient support device for use in an imaging machine.

FIG. 6 is a bottom perspective view an embodiment of a patient support device for use in an imaging machine.

FIG. 7 is an expanded partial view of the embodiment of a patient support device for use in an imaging machine of FIG. 6.

FIGS. 8 and 9 are end and side views of an embodiment of a patient support device for use in an imaging machine mounted to a rigid concave base member and supporting a person.

FIGS. 10, 11, 12, and 13 are end and perspective views of an embodiment of a patient support device for use in an imaging machine mounted to a rigid concave base member and inflated but unloaded (FIG. 10), inflated and loaded with a simulated load (FIG. 11), partially inflated and loaded with a simulated load (FIG. 12), and uninflated and loaded with a simulated load (FIG. 13).

FIG. 14 is a bottom perspective view of another embodiment of a patient support device for use in an imaging machine, including longitudinal side chambers.

FIG. 15 is a partial expanded perspective view of the embodiment of a patient support device for use in an imaging machine of FIG. 14.

FIG. 16 is an end perspective view of the embodiment of a patient support device for use in an imaging machine of FIG. 14.

FIG. 17 is a bottom perspective view of another embodiment of a patient support device for use in an imaging machine, showing a transverse attachment member.

FIG. 18 is another bottom perspective view of the embodiment of FIG. 17, showing a transverse attachment member.

FIG. 19 is an expanded view of a portion of the bottom of the embodiment of FIGS. 17 and 18, showing a friction pad under the transverse attachment member.

### DETAILED DESCRIPTION

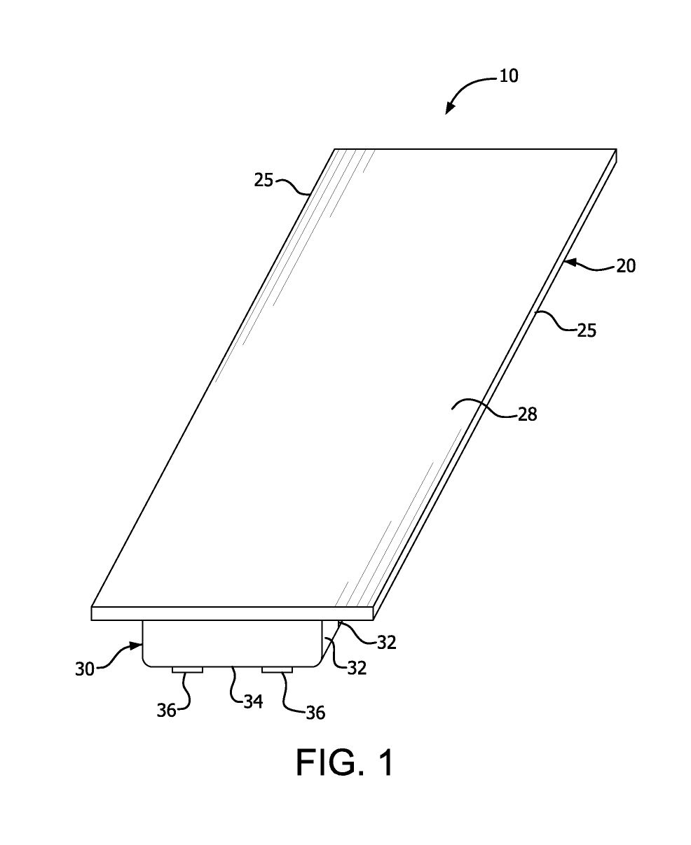

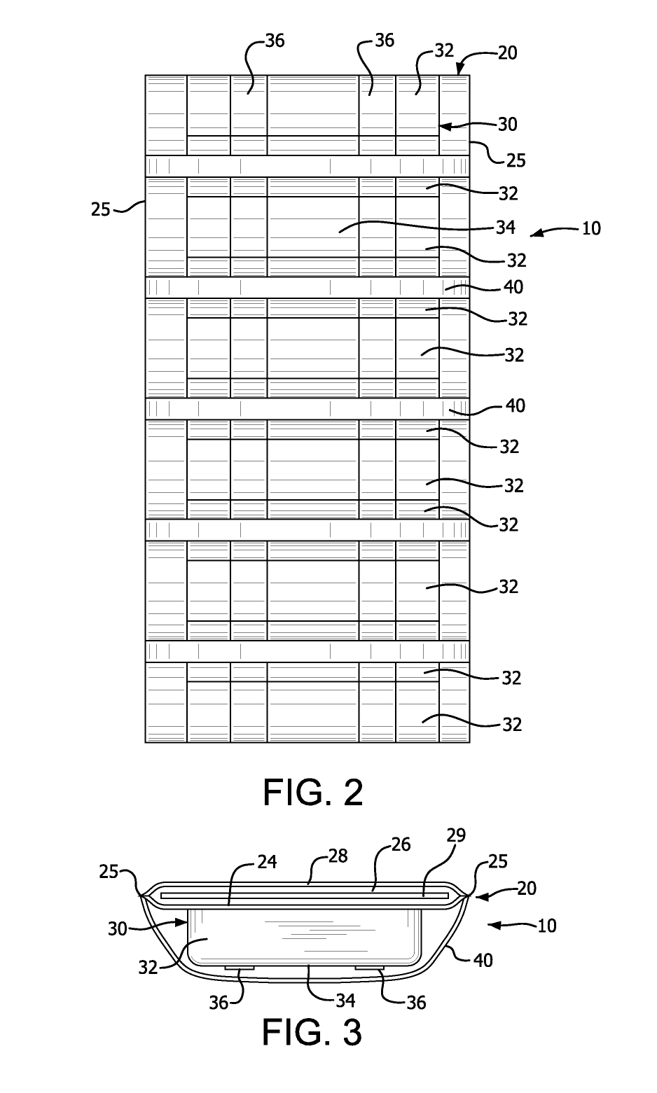

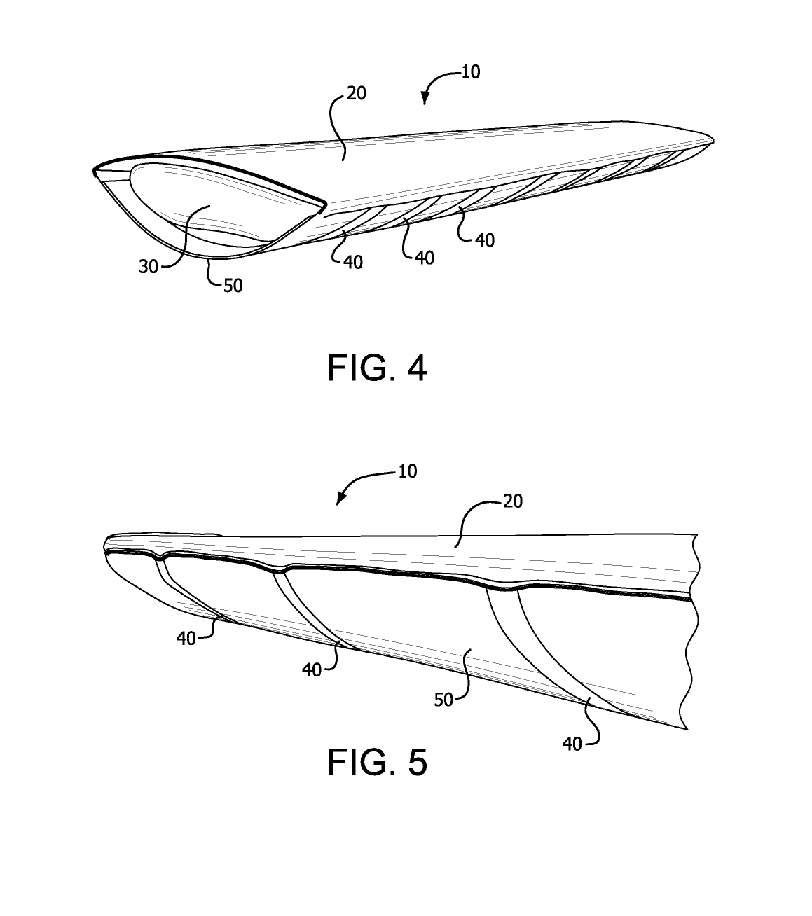

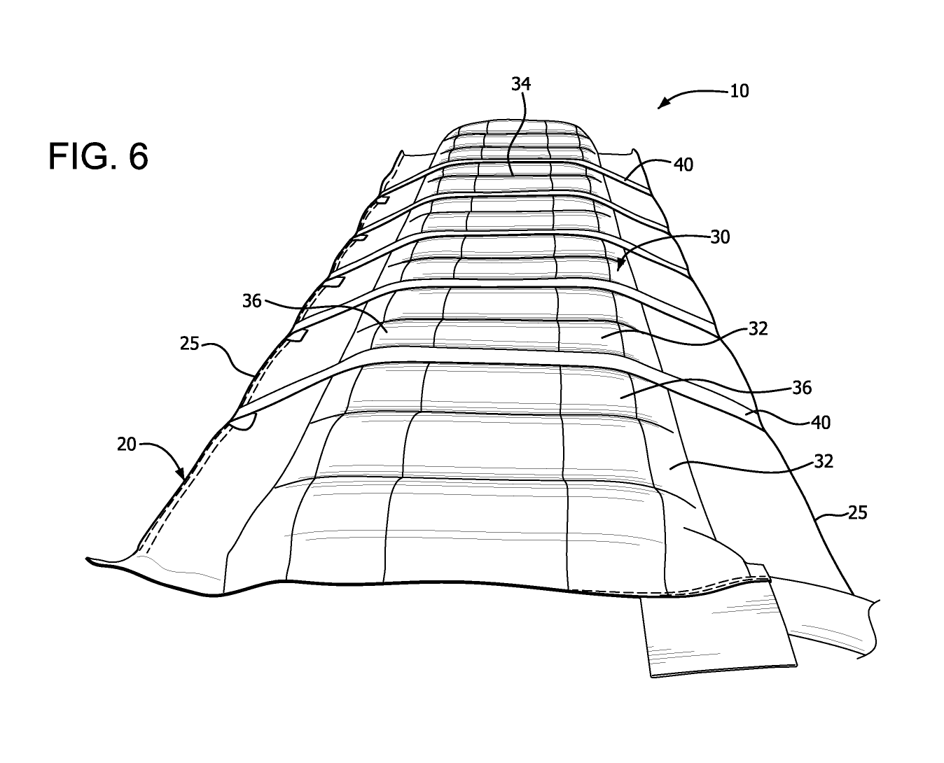

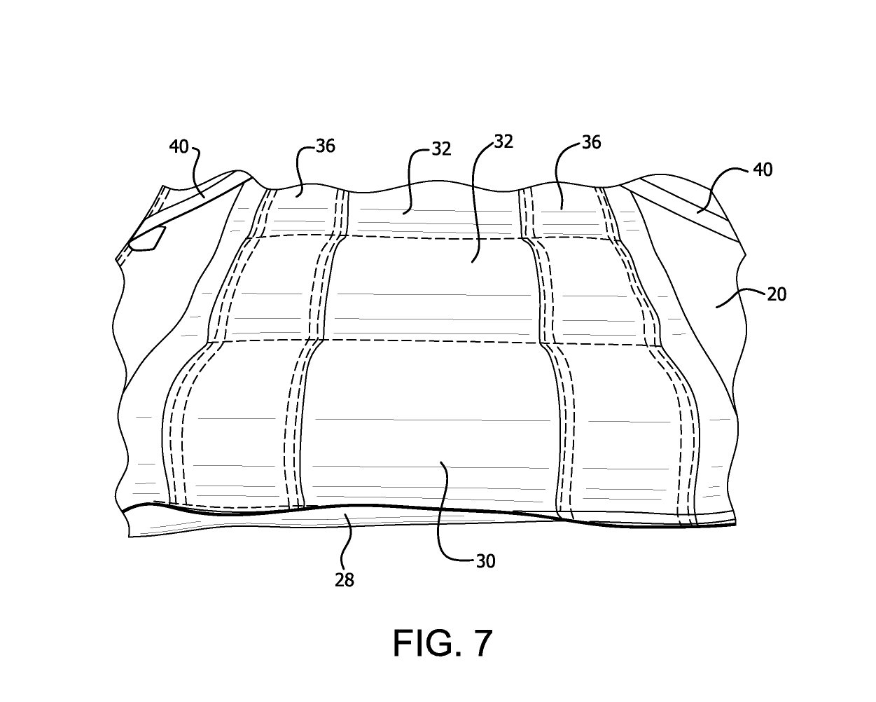

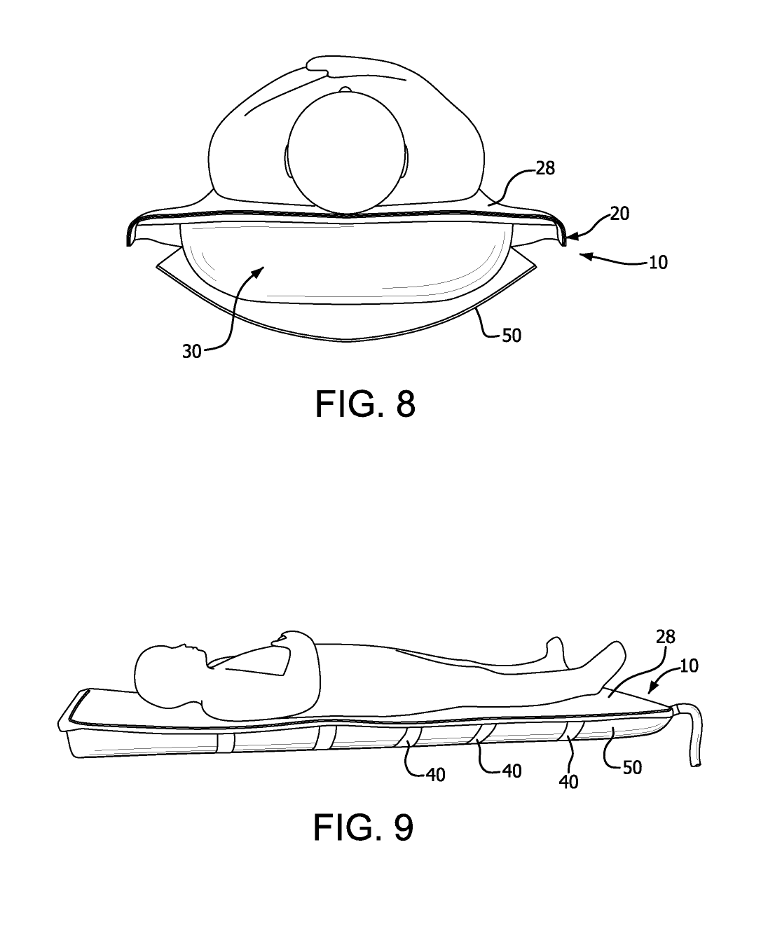

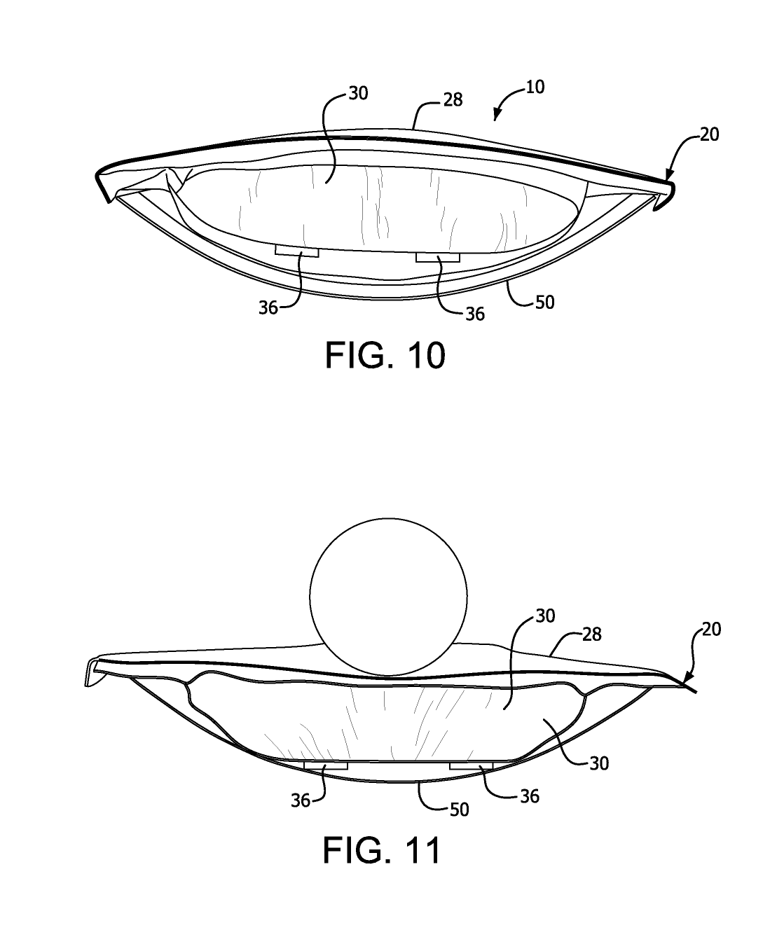

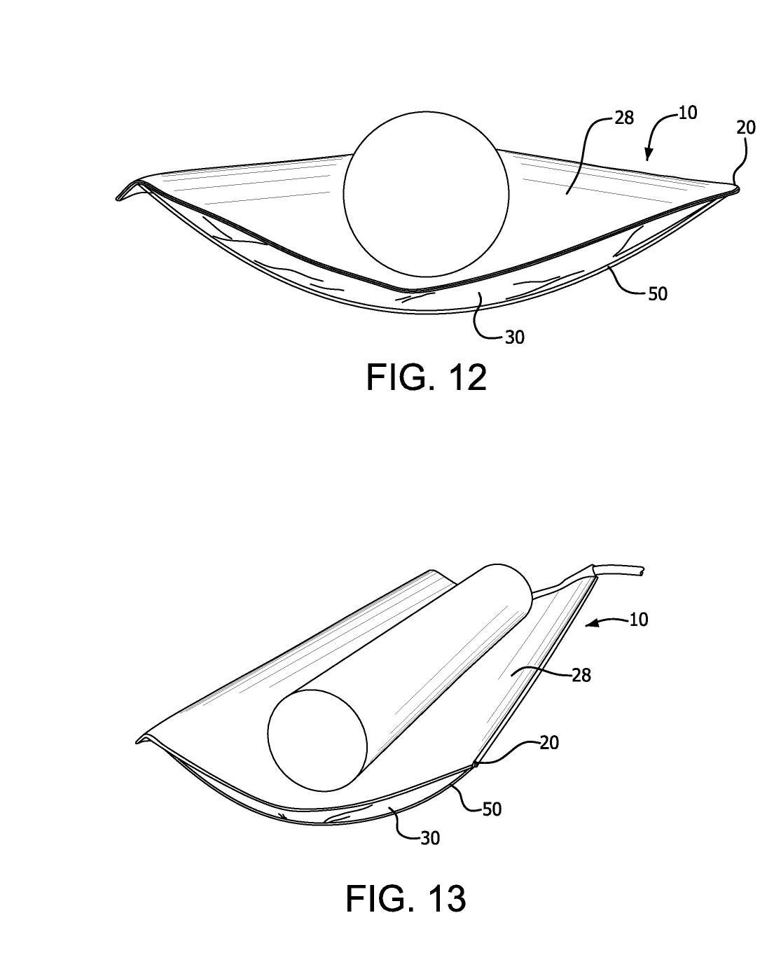

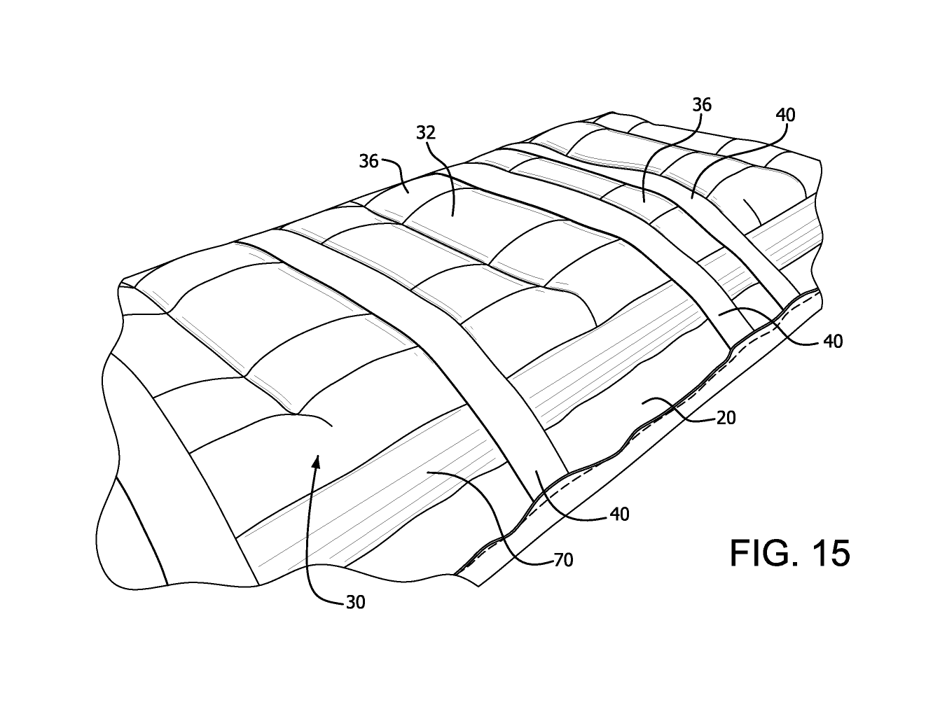

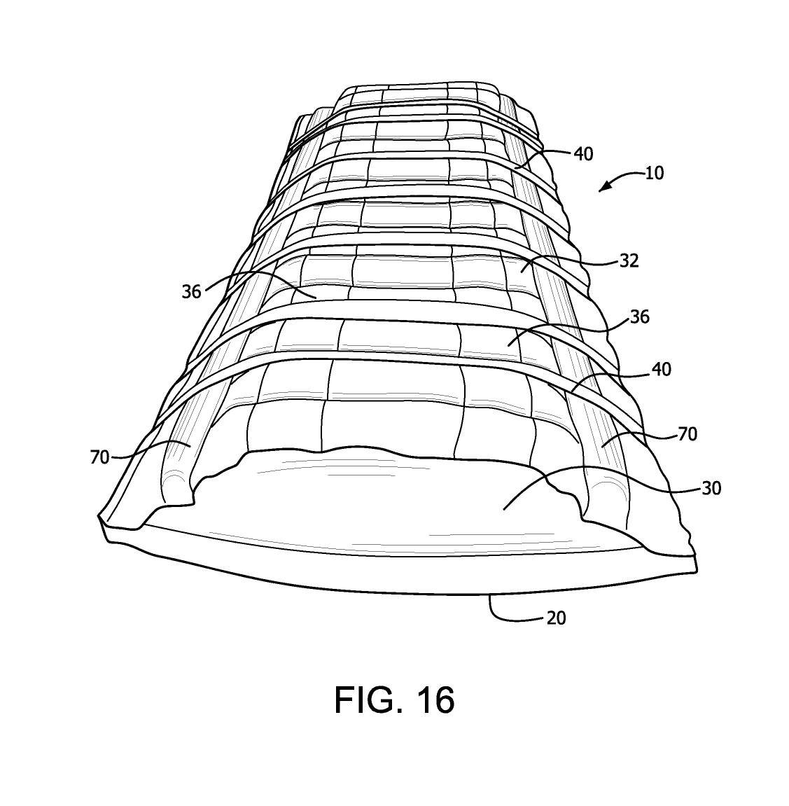

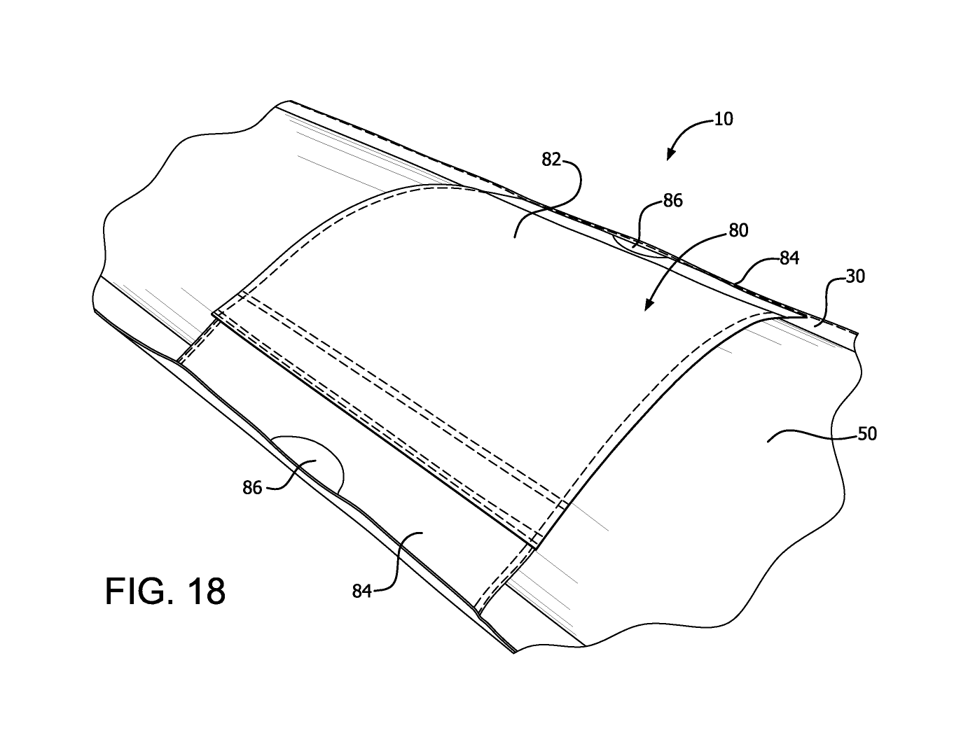

An embodiment of a patient support device 10 is shown in FIG. 1 for use in an imaging machine, and an alternate embodiment of such a device 10 is shown in FIGS. 2 and 3. Various embodiments of the device are shown in FIGS. 4-19.

The device 10 includes an inflatable mattress 30 having a series of transverse air chambers 32. The air chambers 32 are positioned adjacent to one another and interconnected internally so that a single supply of pressurized air to the mattress 30 can be used to uniformly inflate the air chambers 32. Air can be supplied via an inlet at any location of the mattress 30, including but not limited to the head end, the foot end, and the sides. Collectively, the air chambers 32 form a generally rectangular shape, with each air chamber 32 having a width transverse to the longitudinal direction of the mattress 30, the width of the air chambers 32 defining a lateral direction of the mattress 30. The mattress 30 is provided with a pressure control mechanism or venting control mechanism to allow pressurized air to escape in a controlled flow rate from the mattress 30 when the mattress 30 is inflated by an external air source. In one variation, the pressure control mechanism includes the mattress 30 having a perforated, microperforated, or microvented bottom surface 34. In another variation, the pressure control mechanism may include a nonperforated bottom surface 34 in combination with a valve, an orifice, a microporous plug, or the like to provide a regulated rate of air release from the air chambers 32.

A sheet member 20 is disposed across a top of the mattress 30. The sheet member 20 may form a top wall of the air chambers 32 or may be a separate member attached on top of the air chambers 32. The sheet member 20 extends laterally outward with respect to the transverse air chambers 32 and defines a patient support surface 28 on which a patient can be supported.

The device 10 preferably includes a means to secure the inflatable mattress 30 or to inhibit sliding of the inflatable mattress 30 when the device 10 is supported by an underlying base member. For example, as shown in FIG. 4, the device 10 may be supported by a rigid concave base member 50. The base member 50 may be adapted to be fastened to a similarly shaped patient support tray or pallet in an imaging machine.

In one embodiment, a friction enhancing material may be applied to at least a portion of the bottom surface 34 of the inflatable mattress 30. For example, as shown in FIGS. 2, 6, 7, 14, 15, and 16, at least one longitudinal strip of friction enhancing material 36 may be attached to the bottom surface 34 of the inflatable mattress 30 to inhibit sliding of the inflatable mattress 30 when the device 10 is supported by the base member 50.

In another embodiment, the device 10 may include a plurality of fasteners, including but not limited to snaps, buttons, zippers, and hook-and-loop fasteners, to removably fasten the inflatable mattress 30 to the base member 50. The fasteners may be permanently or removably attached to the inflatable mattress 30, and preferably extend laterally outward with respect to the sheet member 20 so as to be accessible when a patient is supported on the device 10. In yet another embodiment, the device 10 may be secured to the base member 50 using a plurality of straps 40 that are fastened to opposite edges 25 of the sheet member 20 and extend beneath the bottom surface 34 of the inflatable mattress 30. In use, the base member 50 would be inserted between the bottom surface 34 and the straps 40, and the base member 50 would be separately secured to an imaging device.

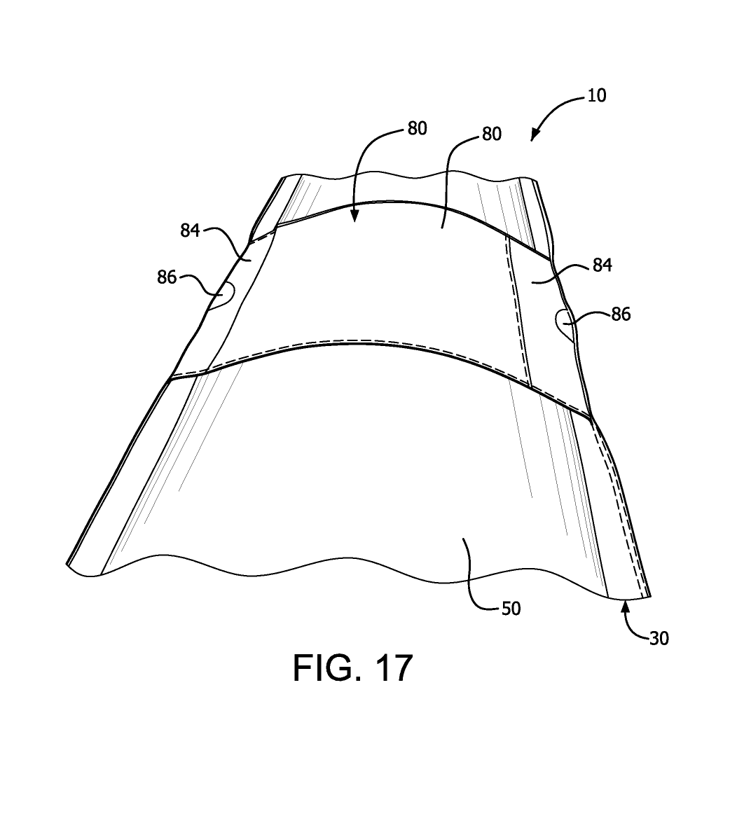

In another embodiment of an inflatable mattress 30, depicted in FIGS. 17-19, a transverse attachment member 80 is provided in place of the straps 40. In the depicted embodiment, the transverse attachment member 80 is extends for a portion of the length of the device 10 and is positioned in a middle section of the length of the device 10. The transverse attachment member 80 secures the mattress 30 to the base member 50 by looping under the base member 50. The transverse attachment member 80 provides a mechanical fastening mechanism, such as hook-and-loop type fasteners or other known fasteners, for securing the device 10 to the pallet of an imaging machine. The transverse attachment member 80 also provides grasping features that enable the device 10 to be readily manipulated by a user, for example to install or remove the device 10 from an imaging machine or to transfer the device 10 from one imaging machine to another.

The transverse attachment member 80 includes a central portion 82 bounded by an attachment portion 84 on each side of the central portion 82. Each attachment portion 84 is attached to a respective edge 25 of the mattress 30. The central portion 82 may be made from a relatively smooth material with a low coefficient of friction, such as a non-stick or low-friction fabric, to enable the device 10 to slide longitudinally along an imaging machine pallet during installation and removal of the device 10. Each attachment portion 84 may include a frictional material for engaging or mating with a frictional material mounted on the upper surface of the imaging machine pallet. In one embodiment, each attachment portion 84 is partially or entirely covered with one feature (typically the softer or loop feature) of a conventional hook-and-loop fastener, so as to enable secure mating with an opposite feature (typically the harder or hook feature) of a hook-and-loop fastener that is mounted on the upper surface of the imaging machine pallet.

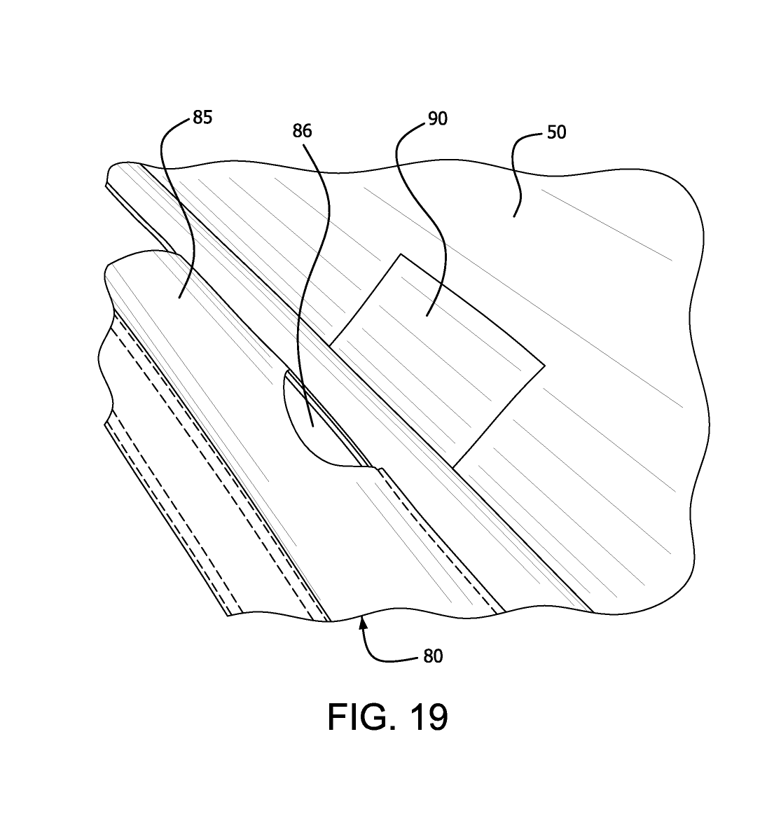

A handhold cutout 86 may be provided in each of the attachment portions 84 to facilitate installation and removal of the device 10. In conjunction, as shown particularly in FIG. 19, a non-slip or high-friction pad or surface 90 is provided on the base member 50 in alignment with each of the handhold cutouts 86. When a user inserts a hand into one of the cutouts 86 to lift the device, the non-slip pad 90 enables the user's hand to achieve a positive frictional grip of the device 10, to facilitate lifting and carrying of the device 10, for example from one imaging machine to another.

In one embodiment, the sheet member 20 of the patient support device 10 is a single sheet of material. In another embodiment, the sheet member 20 of the patient support device 10 includes an upper sheet 22 and a lower sheet 24 forming a sleeve 26 adapted to receive an accessory 29. The sleeve 26 positions the accessory 29 beneath the patient support surface 28. In yet another embodiment, the sheet member 20 need not include a sleeve, but can include fasteners for securing an accessory 29 on top of the patient support surface 28. The accessory 29 can span the entire size of the patient support surface 28, or can be smaller than the patient support surface 28. The accessory 29 may include one or more of a therapeutic pad, a support cushion, and/or a flexible, semi-rigid board that is adapted to flex between a generally flat shape (when the mattress 30 is fully inflated) and a generally curved shape about a longitudinal axis of the patient support surface 28 (when the mattress 30 is deflated and the patient support surface 28 conforms to the base member 50).

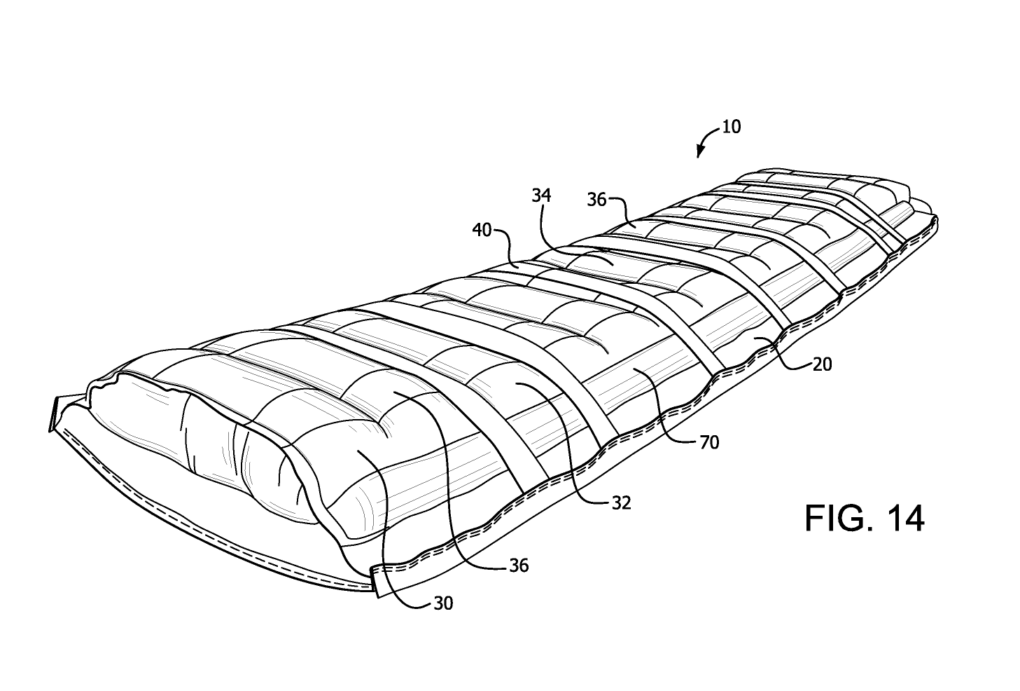

To improve stability, another embodiment of an inflatable mattress 30, as shown in FIGS. 14-16 may include a pair of longitudinal air chambers 70 interconnected with the transverse air chambers 32 and extending longitudinally along at least a portion of either side of the inflatable mattress 30. When the inflatable mattress 30 is supported by a rigid concave base member 50, the longitudinal air chambers 70 fill in a portion of the space on each side of the transverse air chambers between the base member 50 and the upper sheet 22 of the inflatable mattress 30.

A patient support device as disclosed herein can be used to facilitate positioning a patient in an imaging machine. Prior to positioning a patient, a flow of pressurized air is supplied to the inflatable mattress 30 to cause the patient support surface 28 to be maintained in a generally flat condition. Because the inflatable mattress is provided with a pressure control mechanism, a continuous flow of air may be required and/or desirable to maintain the mattress 30 in a regulated, inflated state. In one embodiment, as shown, the bottom sheet 34 of the mattress 30 includes microperforations. In another embodiment, the pressure control mechanism can include a valve, an orifice, a microporous plug, or the like. When the pressure control mechanism is set to allow air to continually escape from the inflatable mattress 30, it is preferable to use a source of pressurized air for which the flow rate of air can be controlled as desired to increase or decrease the pressure in the mattress 30.

Next, a supine patient is positioned on top of the patient support surface 28 while continuing to supply the flow of pressurized air at a sufficient level to maintain the patient support surface 28 in a generally flat condition. The patient may be transferred from a stretcher or gurney onto the device 10 which is already positioned on the base member 50, or an ambulatory patient may simply lie down on top of the device 10. Alternatively, the device 10 itself, in an embodiment preferably having a microperforated bottom sheet 34, can be used as a patient transfer device—the patient can remain supine on the device 10 while the device 10, with the patient, is caused to slide laterally from the stretcher or gurney onto the concave rigid base member 50 (which is itself supported in an imaging machine or other fixture capable of supporting the weight of the patient).

Once the patient is positioned as desired on the patient transfer surface 28, the flow of pressurized air is decreased (while air continues to escape from the microperforated bottom surface 34 of the mattress 30) such that, under the weight of the patient, the patient support surface 28 is lowered. Eventually, as most of the air has been forced out of the mattress 30, the patient support surface 28 generally conforms to the shape of the concave rigid base member 50 and the patient is supported in position for imaging.

When imaging is complete, the patient is raised up by increasing the flow of pressurized air to overcome the air escaping from the pressure control mechanism (e.g., the microperforated bottom surface 34 of the mattress 30) such that the patient support surface 28 is raised to a generally flat condition, supporting the patient above the concave rigid base member 50.

While the invention has been disclosed with reference to certain preferred embodiments, numerous modifications, alterations, and changes to the described embodiments are possible without departing from the sphere and scope of the invention, as defined in the appended claims and equivalents thereof.

1. A patient support device for use in conjunction with a pallet of an imaging machine, comprising:
an inflatable mattress having a plurality of adjacent interconnected transverse air chambers and a mechanism for controlled venting of the air chambers;
a sheet member disposed across a top of the mattress and extending laterally outward with respect to the transverse air chambers, the sheet member defining a patient support surface; and
at least one longitudinal strip of friction enhancing material attached to a bottom surface of the inflatable mattress to inhibit sliding of the inflatable mattress on the pallet.

2. The patient support device of claim 1, wherein the mechanism for controlled venting of the air chambers includes a microperforated bottom surface on the inflatable air mattress.

3. The patient support device of claim 1, wherein the mechanism for controlled venting of the air chambers includes a partially or entirely perforated bottom surface on the inflatable mattress.

4. The patient support device of claim 1, wherein the mechanism for controlled venting of the air chambers includes a nonperforated bottom surface on the inflatable mattress and a valve to selectively allow air to be passively or actively removed for deflation.

5. The patient support device of claim 1, wherein the imaging machine includes a rigid base member configured to mate with the pallet, the device further comprising:
a plurality of straps each fastened to opposite edges of the sheet member and extending beneath the bottom surface of the inflatable mattress for securing the inflatable mattress to the rigid base member.

6. The patient support device of claim 1, wherein the imaging machine includes a rigid base member configured to mate with the pallet, the device further comprising:
a transverse attachment member fastened to opposite edges of the sheet member and extending beneath the bottom surface of the inflatable mattress, the transverse attachment member including attachment portions at each end thereof adjacent to the edges of the sheet member, for securing the inflatable mattress to the rigid base member.

7. The patient support device of claim 6, wherein the transverse attachment member includes a central low-friction portion bounded on each side near the edges of the sheet member by an attachment portion, each attachment portion including an attachment feature configured to mate with a corresponding feature on the pallet of the imaging machine.

8. The patient support device of claim 6, wherein the base member includes a high-friction pad, the transverse attachment member of the device further comprising a handhold cutout in each attachment portion for accessing the high-friction pad on the rigid base member.

9. The patient support device of claim 1, the sheet member including a sleeve for positioning an accessory beneath the patient support surface.

10. The patient support device of claim 1, further comprising an accessory attachable to the top of the patient support surface of the sheet member using fasteners.

11. The patient support device of claim 10, wherein the fasteners include one or more of snaps, buttons, hook-and-loop fasteners, and zippers.

12. The patient support device of claim 1, the inflatable mattress further including one or more longitudinal air chambers interconnected with the transverse air chambers and extending longitudinally along at least a portion of either side of the inflatable mattress, to provide increased stability of the inflatable mattress when inflated.

13. The patient support device of claim 12, comprising a pair of longitudinal air chambers, with one air chamber on one side of the transverse air chambers and the other air chamber on the other side of the transverse air chambers.

14. A patient support device for use in conjunction with a defined space pallet of an imaging machine and a rigid base member configured to mate with the pallet, comprising:
an inflatable mattress having a plurality of adjacent interconnected transverse air chambers and a mechanism for controlled venting of the air chambers;
a sheet member disposed across a top of the mattress and extending laterally outward with respect to the transverse air chambers, the sheet member defining a patient support surface;
two longitudinal strips of friction enhancing material attached to a bottom surface of the inflatable mattress to inhibit sliding of the inflatable mattress; and
a mechanism for securing the patient support device to the rigid base member.

15. The patient support device of claim 14, wherein the mechanism for securing the patient support device to the rigid base member includes a plurality of straps each fastened to opposite edges of the sheet member and extending beneath the bottom surface of the inflatable mattress.

16. The patient support device of claim 14, wherein the mechanism for securing the patient support device to the rigid base member includes a transverse attachment member fastened to opposite edges of the sheet member and extending beneath the bottom surface of the inflatable mattress, the transverse attachment member including attachment portions at each end thereof adjacent to the edges of the sheet member.

17. The patient support device of claim 16, wherein the transverse attachment member includes a central low-friction portion bounded on each side near the edges of the sheet member by an attachment portion, each attachment portion including an attachment feature configured to mate with a corresponding feature on the pallet of the imaging machine.

18. The patient support device of claim 16, wherein the base member includes a high-friction pad, the transverse attachment member further comprising a handhold cutout in each attachment portion for accessing thea high-friction pad on the base member.

19. The patient support device of claim 14, the sheet member including a sleeve for positioning an accessory beneath the patient support surface.

20. The patient support device of claim 14, further comprising fasteners for attaching an accessory on top of the patient support surface.

21. The patient support device of claim 14, wherein the mechanism for controlled venting of the air chambers includes the inflatable mattress having a microperforated bottom surface.

22. The patient support device of claim 14, wherein the mechanism for controlled venting of the air chambers includes the transverse air chambers having a substantially air-tight bottom surface, and a pressure control valve for controlled release of air from the inflatable mattress.

23. A method for positioning a patient on a pallet of an imaging machine using a patient support device including an inflatable mattress having a plurality of adjacent interconnected transverse air chambers and a mechanism for controlled venting of the air chambers; a sheet member disposed across a top of the mattress and extending laterally outward with respect to the transverse air chambers, the sheet member defining a patient support surface; and at least one longitudinal strip of friction enhancing material attached to a bottom surface of the inflatable mattress to inhibit sliding of the inflatable mattress, the method comprising:
positioning the patient support device on the pallet;
supplying a flow pressurized air to the inflatable mattress to cause the patient support surface to be maintained in a generally flat condition;
positioning a patient on top of the patient support surface while continuing to supply the flow of pressurized air at a sufficient level to maintain the patient support surface in a generally flat condition; and
decreasing the flow of pressurized air while allowing air to escape from the mechanism for controlled venting such that, under the weight of the patient, the patient support surface is lowered so as to generally conform to the shape of the pallet, thereby lowering the patient into position for imaging.

24. The method of claim 23, wherein the mechanism for controlled venting includes the inflatable mattress having a microperforated bottom surface.

25. The method of claim 23, further comprising increasing the flow of pressurized air to overcome the air escaping from the mechanism for controlled venting such that the patient support surface is raised to a generally flat condition, thereby supporting the patient above the pallet.

26. The method of claim 23, wherein the imaging machine includes a concave rigid base member configured to mate with the pallet, wherein the device further includes a plurality of straps each fastened to opposite edges of the sheet member and extending beneath the bottom surface of the inflatable mattress for securing the patient support device to the base member.

27. The method of claim 23, wherein the imaging machine includes a concave rigid base member configured to mate with the pallet, wherein the device further includes a transverse attachment member fastened to opposite edges of the sheet member and extending beneath the bottom surface of the inflatable mattress for securing the patient support device to the base member, the transverse attachment member including attachment portions at each end thereof adjacent to the edges of the sheet member.
